(19) United States
(12) Patent Application Publication
(10) Pub. No.: US 2024/0023693 A1
(43) Pub. Date: Jan. 25, 2024

(54) MAGNETIC NAIL ASSEMBLY, SYSTEM, AND METHOD

(71) Applicant: Audrey Bakerson, Niles, MI (US)

(72) Inventor: Audrey Bakerson, Niles, MI (US)

(21) Appl. No.: 18/353,067

(22) Filed: Jul. 15, 2023

Related U.S. Application Data
(60) Provisional application No. 63/390,462, filed on Jul. 19, 2022.

(51) Int. Cl. A45D 31/00; A61K 8/19; A61Q 3/02

(52) U.S. Cl. CPC A45D 31/00; A61K 8/19; A61Q 3/02; A61K 2800/47

(57) ABSTRACT

A magnetic nail system includes an artificial nail having an upper face and an opposing under surface; a first magnetic element to be applied to a fingernail or toenail; and a second magnetic element provided on the artificial nail to be magnetically attracted to the first magnetic element. The artificial nail adheres to the fingernail or toenail by magnetic attraction between the first magnetic element and the second magnetic element. The first magnetic element and the second magnetic element include either a permanent magnet, a magnetic strip, or a magnetic slurry having magnetic particles suspended in a mixture with a drying liquid, polymer material or the magnetic slurry particles are suspended in a nail polish material.

### CROSS-REFERENCE TO RELATED APPLICATIONS

This Application claims priority to and the benefit of U.S. Provisional Patent Application No. 63/390,462, titled “MAGNETIC NAIL ASSEMBLY, SYSTEM, AND METHOD,” and filed on Jul. 19, 2022, the disclosure of which is incorporated herein by reference in its entirety for all purposes.

### TECHNICAL FIELD

The present disclosure relates generally to the field of decorative artificial nails.

### DESCRIPTION OF RELATED ART

Artificial nails or press-on nails are commonly used to provide a quick solution for a desired aesthetic or visual effect to fingernails. Prior to artificial nails, a person may need to wait for fingernails to grow to a desired length or apply a design directly to the surface of a fingernail. These methods required time and often the expertise of a trained technician. Artificial nails allowed users to quickly obtain a professional design without needing specialty equipment or skills.

However, artificial nails have several significant drawbacks. For example, artificial nails are typically applied to a fingernail using an adhesive which should be strong enough to hold the artificial nail to the fingernail. When the adhesive fails, the artificial nail no longer functions as intended.

Adhesives used in artificial nails are typically single use. If a nail is incorrectly applied, mispositioned, or fails to yield a desired aesthetic effect, the artificial nail must be removed by destroying the adhesive.

Removing an artificial nail may challenging even when applied correctly. Often it may need to be physically broken or soaked in chemicals to facilitate removal such as acetone, ethyl acetate, butyl acetate, and propylene carbonate. These chemicals are known to cause skin irritation and still may fail to fully remove any adhesive material on the fingernail.

WO2022/056541 to Tuttle discloses a magnetic artificial nail which couples to a fingernail by magnetic attraction to an ancillary base structure that must be secured to the surface of a fingernail. However, this solution still fails to provide a simplified and secure method of attaching an artificial nail. The base must still be secured to a fingernail using adhesive. Therefore, the base would still possess the same shortfalls of any other artificial nail in that the base may fall off if the adhesive fails. Additionally, if the base is applied incorrectly, it cannot be easily adjusted.

What is needed is an artificial nail that is easy to apply and remove without damaging skin or natural fingernails. What is needed is an artificial nail that may be easily and quickly interchanged to achieve desired designs.

### BRIEF SUMMARY

In one aspect, a magnetic nail system includes (a) an artificial nail having an upper face and an opposing under surface, (b) a first magnetic element configured to be applied to a fingernail, (c) a second magnetic element provided on the under surface of the artificial nail configured to be magnetically attracted to the first magnetic element, and where, the artificial nail is configured to adhere to the fingernail by magnetic attraction between the first magnetic element and the second magnetic element.

The magnetic nail system may also include where the first magnetic element, the second magnetic element, or both include a permanent magnet.

The magnetic nail system may also include where the first magnetic element, the second magnetic element, or both include a magnetic strip.

The magnetic nail system may also include where the first magnetic element, the second magnetic element, or both include a magnetic slurry having magnetic particles suspended in a mixture with a drying liquid or polymer material.

The magnetic nail system may also include where the artificial nail includes a plurality of holes configured to receive a corresponding magnetic element therein.

The magnetic nail system may also include where the first magnetic element is a clear nail polish having magnetic particles distributed therein.

The magnetic nail system may also include where the first magnetic element includes either a permanent magnet, a magnetic strip, or a magnetic slurry having magnetic particles suspended in a mixture with a drying liquid, polymer material or the magnetic slurry particles are suspended in a nail polish material, and where the second magnetic element includes either a permanent magnet, a magnetic strip, or a magnetic slurry having magnetic particles suspended in a mixture with a drying liquid, polymer material or the magnetic slurry particles are suspended in a nail polish material.

A method for applying the magnetic nail assembly may also include includes (a) providing the first magnetic element in a nail polish to form a magnetic nail polish, (b) applying the magnetic nail polish to the surface of a fingernail or toenail, (c) allowing the magnetic nail polish to dry or cure, and (d) placing the under surface of the artificial nail on top of the magnetic nail polish applied to the fingernail or toenail.

A magnetic nail system includes a plurality of artificial nails according to claim 1, where each of the plurality of artificial nails corresponds to a different sized fingernail including a thumb fingernail, an index finger fingernail, a middle finger fingernail, a ring finger fingernail, and a pinky fingernail. The the first magnetic element, the second magnetic element, or both may include a permanent magnet, a magnetic strip, or a magnetic slurry, or a combination thereof. The magnetic slurry can include nail polish. Other technical features may be readily apparent to one skilled in the art from the following figures, descriptions, and claims.

The magnetic nail system may also include where the magnetic slurry particles are suspended in a nail polish material.

The magnetic nail system may also include where the first magnetic element includes the magnetic slurry particles suspended in a nail polish and the second magnetic element includes a magnetic strip.

The magnetic nail system may also include where the artificial nail includes a design element.

The magnetic nail system may also include where the artificial nail includes a color design.

The magnetic nail system may also include where the the magnetic slurry particles are ground to a fine coarseness, medium coarseness, or rough coarseness.

For purposes of summarizing the disclosure, certain aspects, advantages, and novel features of the disclosure have been described herein. It is to be understood that not necessarily all such advantages may be achieved in accordance with any one particular embodiment of the disclosure. Thus, the disclosure may be embodied or carried out in a manner that achieves or optimizes one advantage or group of advantages as taught herein without necessarily achieving other advantages as may be taught or suggested herein. The features of the disclosure which are believed to be novel are particularly pointed out and distinctly claimed in the concluding portion of the specification. These and other features, aspects, and advantages of the present disclosure will become better understood with reference to the following drawings and detailed description.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates an exemplary magnetic artificial nail according to the present disclosure.

FIG. 2 illustrates plurality magnetic artificial nails according to the present disclosure.

FIG. 3 is a schematic drawing of a plurality of artificial nails with unique design features for application on a magnetic nail polish.

The various embodiments of the present disclosure will hereinafter be described in conjunction with the appended drawings, wherein like designations denote like elements.

FIG. 4 illustrates an exemplary artificial nail system having holes for magnets according to the present disclosure.

FIG. 5 illustrates a process 500 in accordance with the present disclosure.

### DETAILED DESCRIPTION

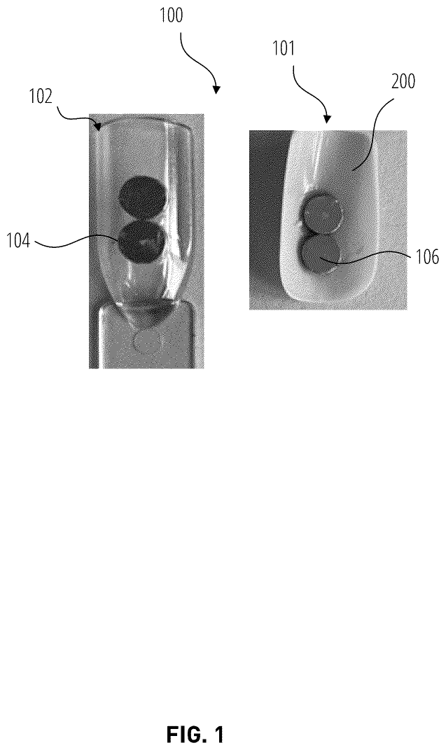

Referring to FIGS. 1-5, the present disclosure relates to a magnetic artificial nail assembly and system. The nail system can be applied to fingernails or, by extension toenails. The term “magnet” can include a magnet of various forms, such as a strip magnet, magnetic tape, a magnetic disc, a rectangular magnet, a liquid magnet or magnetic slurry, or any combination thereof. The term “magnetic material” includes any material exhibiting magnetic properties according to the description above, but also can include any material attracted to or repelled by a magnet such as, but not limited to, iron, steel, nickel, or cobalt. Magnetic properties can be any ferrous iron properties or metallic metal. The term “magnet” or “magnetic material” can also include magnetic liquid, rare earth magnets, flexible magnets, neodymium magnets or any combination thereof.

In an example, a magnetic system 100 is shown having an artificial nail 101 configured to be mounted on a fingernail 102. Fingernail 102 is shown as representative of an actual fingernail of a user to illustrate the functional concepts. In this example, a surface representing a first magnetic element 104 is provided on an upper face of fingernail 102. Artificial nail 101 includes a corresponding second second surface forming a second magnetic element 106 on an under surface 200. In an example, first magnetic element 104 and second magnetic element 106 are configured to be magnetically attracted to one another due to their opposite polarities, thus securing artificial nail 101 to fingernail 102. When artificial nail 101 engages with fingernail 102, magnetic forces between the first magnetic element 104 and second magnetic element 106 secure the two surfaces to be magnetically connect to each other, thus making artificial nail 101 removably attached to fingernail 102.

Artificial nail 101 may be shaped to correspond to a fingernail 102. In an example, artificial nail 101 may be shaped to correspond to a nail of a(n) thumb, index finger, middle finger, ring finger, and/or pinky finger. Artificial nail 101 may also be contoured for an aesthetic purpose which may include matching the contour of fingernail 102. Artificial nail 101 may be formed to match the size of fingernail 102 or artificial nail 101 may be configured such that a portion of its length extends out and beyond the edge of fingernail 102. Artificial nail 101 can be comprised of various materials, including but not limited to, gel, plastic, metal, and the like.

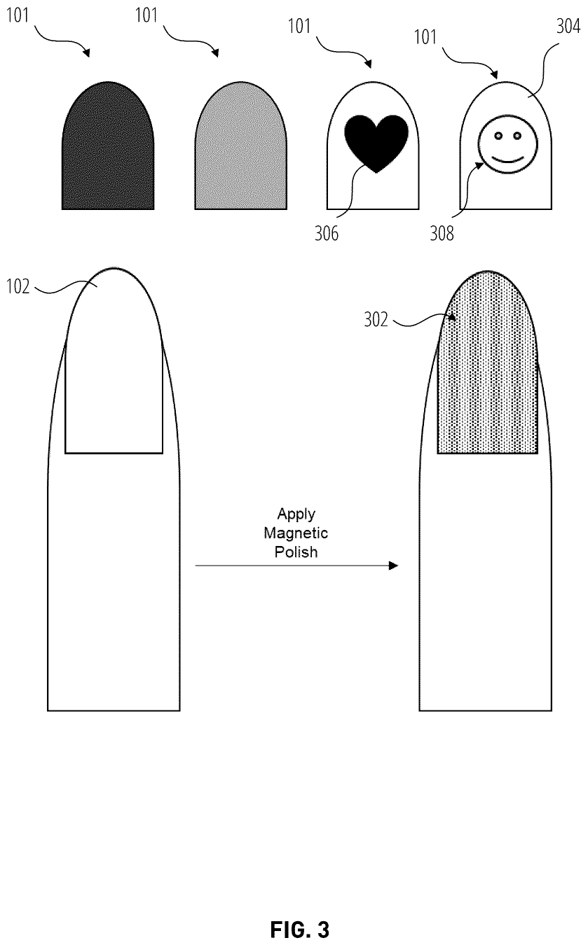

Artificial nail 101 can include an upper face (see upper face 304 on FIG. 3) and an opposing under surface 200. The upper face 304 can be configured to allow for placement of design features such as nail polish or other artistic concepts. Under surfaces 200 includes a first magnetic element 104 to adhere to fingernail 102. First magnetic element 104 can be one or more magnets or made from magnetic material and can be attached to or integral to under surface 200. In an example, first magnetic element 104 is a magnet which may be attached by an adhesive such as glue. First magnetic element 104 may also be configured to match the contour of artificial nail 101. The material used for first magnetic element 104 can define a color that would serve as a polished nail if the artificial nail(s) were to be removed. This can be accomplished by providing multiple base colors of the material or the ability to overlay a nail polish color application that would not affect the magnetic attraction to the artificial nail. A magnetic liquid would also be a consistency that would provide a smooth surface to mimic a colored nail.

In yet another example, the magnetic element can be dispersed in a liquid, like a nail polish and then applied to a nail in a liquid form before drying. This would leave a magnetic surface on the nail. The magnetic particles dispersed, suspended, or dissolved in the applicable liquid should be done evenly to ensure a well dispersed magnetic effect.

In another example, first magnetic element 104 is a permanent magnet and second magnetic element 106 includes a magnetic material such as iron. Alternatively, second magnetic element 106 may be a permanent magnet and first magnetic element 104 includes a magnetic material such as iron, or both first magnetic element 104 and second magnetic element 106 include iron. An advantage of including a magnetic material for either both magnetic elements is that magnetic material or a magnetic slurry may be suspended in a polymer solution, such as nail polish, that can be applied to a fingernail. This allows removal of the magnetic material just as similar to the removal of a conventional nail polish.

Several examples of a magnetic slurry or application of a fast-acting drying liquid or polymer material that includes a magnetic material suspended therein are shown in which a magnetic metal, like iron, is ground into a powder and suspended in nail polish. The solution can be applied to fingernail 102 and allowed to dry. Once dry, a corresponding artificial nail 101 providing a magnet 106 is placed on the fingernail 102.

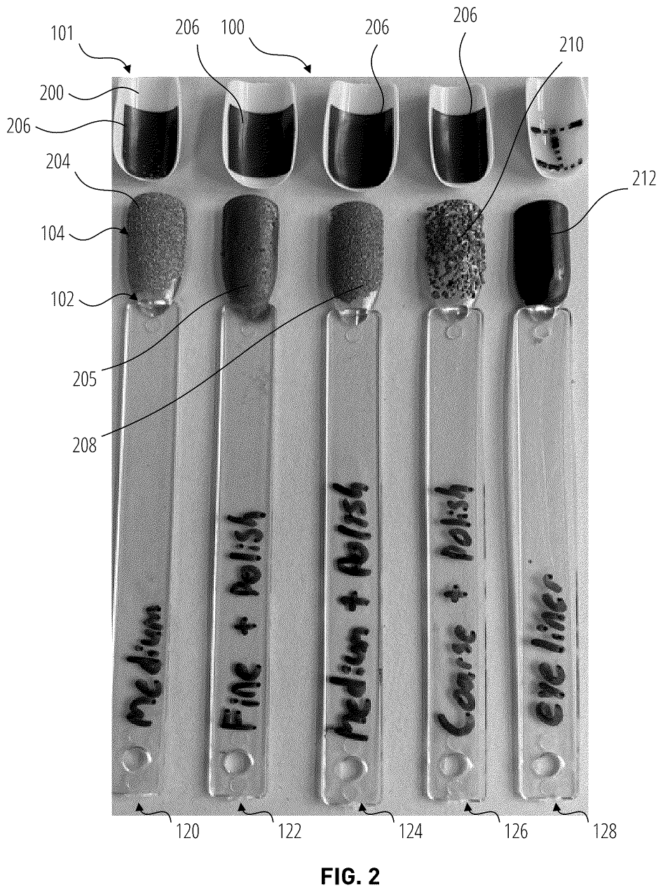

FIG. 2 shows a plurality of mixed magnetic material with a drying liquid polymer or solution. In one example, a medium grain magnetic material is suspended in adhesive to form a medium mixing solution as shown by the example of medium solution stick 120. Medium solution stick 120 shows a stick having a medium coarse solution 204 applied to a fingernail 102. Once dry, artificial nail 101 may be placed on top of fingernail 102. Artificial nail 101 is adhered to fingernail 102 by magnetic attraction between magnetic strip 206 and magnetic material suspended in solution of the first magnetic element 104.

In another example, grounded magnetic material is suspended in nail polish to be applied to a nail (see 204, 205, 208, 210, 212). The magnetic material may be any material with inherent magnetic properties. The magnetic material may be finely ground into a fine coarse polish 205 and applied to a fingernail 102 as shown on fine coarse polish stick 122. The fine coarse material is mixed with a polish to form fine coarse polish 205. In another example, medium course solution is mixed with polish to form a medium coarse polish 208 and applied to the medium course polish stick 124. In still another example, a coarsely ground material can be mixed with polish to form a rough coarse polish 210 as shown on coarse polish stick 126. Eyeliner stick 128 includes a nail 102 with an eyeliner material mixed with magnetic material to form eyeliner material 212.

The mixtures (i.e., 120, 122, 124, or 126) may then be applied to a fingernail 102 and allowed to dry. Once the suspension is dried, artificial nail 101 may then be placed on top of fingernail 102 with its under surface 200 in contact with the dried suspension arranged on the upper face of fingernail 102. The under surface 200 with magnetic element 106 magnetically attracts the to the to the dried solution provided on top of fingernail 102, thus removably attaching artificial nail 101 to fingernail 102.

An advantage of suspending magnetic material in nail polish is that the magnetic solution may be removed by common nail polish removers. Additionally, suspending the magnetic material within a solution allows for application of the suspension using conventional nail polish application techniques without requiring any additional components or methods.

A further advantage of the magnetically removable artificial nail is customizability. In the example of FIG. 3, magnetic polish 302 is applied to fingernail 102. Once magnetic polish 302 is dried or cured, an artificial nail 101 may be applied. Artificial nail 101 is configured to provide a magnetic element 106 on its under surface 200 with optional colors and/or an optional design element (for example heart design element 306 or happy face design element 308) on its opposite upper face 304. Artificial nail 101 may be placed on fingernail 102 such that the magnetic element on its lower face adheres to fingernail 102 by magnetic attraction between the magnetic element and magnetic polish 302.

The adhesion between the artificial nail and magnetic nail polish 304 allows for a method of easily changing artificial nails, without sacrificing any adhesive or causing damage to the fingernail 102 or artificial nail 101. A set of varying designed artificial nails 101 may be included with magnetic polish 302 such that an artificial nail 101 with a first design may be easily removed and replaced with another artificial nail 101 of a second design. Magnetic attraction between two magnetic elements of sufficient power can be separated by basic manual force while maintaining a connection in the absence of such force.

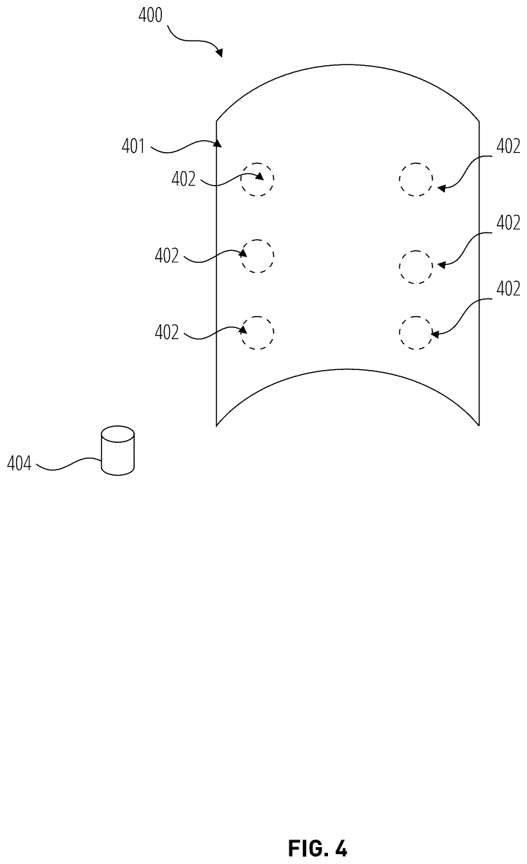

Referring to FIG. 4, another example magnetic system 400 is shown. In this example, artificial nail 401 is formed having a plurality of spaced apart holes 402. In this example, two vertical rows of three holes 402 each are defined. A corresponding magnet 404 is configured to fit into each of the holes 402. Artificial nail 401 is shown on a backside view.

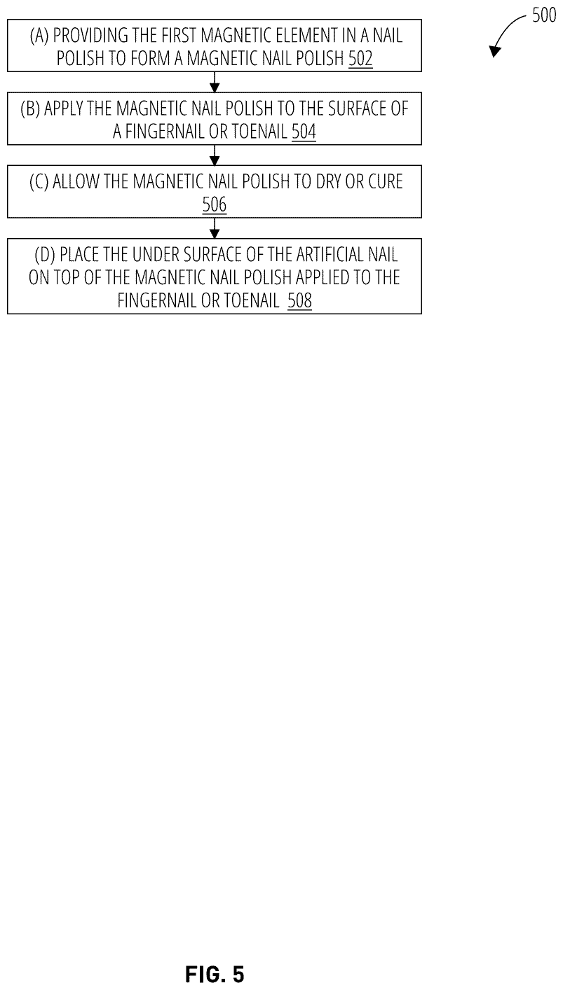

Referring to FIG. 5, an example flowchart illustrating the process 500 of applying a magnetic nail polish of the present disclosure is shown. In block 502, the process includes providing the first magnetic element in a nail polish to form a magnetic nail polish. In block 504, the process applies the magnetic nail polish to the surface of a fingernail or toenail. In block 506, the process continues to allow the magnetic nail polish to dry or cure. In block 508, the process places the under surface of the artificial nail on top of the magnetic nail polish applied to the fingernail or toenail.

It should be noted that the steps described in the method of use can be carried out in many different orders according to user preference. The use of “step of” should not be interpreted as “step for”, in the claims herein and is not intended to invoke the provisions of 35 U.S.C. § 112 (f). Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination, or addition of certain steps, including or excluding certain maintenance steps, etc., may be sufficient.

What is claimed is:

1. A magnetic nail system comprising:
(a) an artificial nail having an upper face and an opposing under surface;
(b) a first magnetic element configured to be applied to a fingernail or toenail;
(c) a second magnetic element provided on the under surface of the artificial nail configured to be magnetically attracted to the first magnetic element; and
wherein, the artificial nail is configured to adhere to the fingernail by magnetic attraction between the first magnetic element and the second magnetic element.

2. The magnetic nail system of claim 1, wherein the first magnetic element, the second magnetic element, or both include a permanent magnet.

3. The magnetic nail system of claim 1, wherein the first magnetic element, the second magnetic element, or both include a magnetic strip.

4. The magnetic nail system of claim 1, wherein the first magnetic element, the second magnetic element, or both include a magnetic slurry having magnetic particles suspended in a mixture with a drying liquid or polymer material.

5. The magnetic nail system of claim 4, wherein the magnetic slurry particles are suspended in a nail polish material.

6. The magnetic nail system of claim 5, wherein the first magnetic element includes the magnetic slurry particles suspended in a nail polish and the second magnetic element includes a magnetic strip.

7. The magnetic nail system of claim 6, wherein the artificial nail includes a design element.

8. The magnetic nail system of claim 6, wherein the artificial nail includes a color design.

9. The magnetic nail system of claim 5, wherein the magnetic slurry particles are ground to a fine coarseness, medium coarseness, or rough coarseness.

10. The magnetic nail system of claim 1, wherein the artificial nail includes a plurality of holes configured to receive a corresponding magnetic element therein.

11. The magnetic nail system of claim 1, wherein the first magnetic element is a clear nail polish having magnetic particles distributed therein.

12. The magnetic nail system of claim 1, wherein the first magnetic element includes either a permanent magnet, a magnetic strip, or a magnetic slurry having magnetic particles suspended in a mixture with a drying liquid, polymer material or the magnetic slurry particles are suspended in a nail polish material, and
wherein the second magnetic element includes either a permanent magnet, a magnetic strip, or a magnetic slurry having magnetic particles suspended in a mixture with a drying liquid, polymer material or the magnetic slurry particles are suspended in a nail polish material.

13. A method for applying the magnetic nail assembly of claim 1 comprising:
(a) providing the first magnetic element in a nail polish to form a magnetic nail polish;
(b) applying the magnetic nail polish to the surface of a fingernail or toenail;
(c) allowing the magnetic nail polish to dry or cure; and
(d) placing the under surface of the artificial nail on top of the magnetic nail polish applied to the fingernail or toenail.

14. A magnetic nail system comprising a plurality of artificial nails according to claim 1, wherein each of the plurality of artificial nails corresponds to a different sized fingernail or toenail including a thumb fingernail or big toenail, an index finger fingernail or second toenail, a middle finger fingernail or middle toenail, a ring finger fingernail or fourth toenail, and a pinky fingernail or pinky toenail, and wherein the first magnetic element, the second magnetic element, or both include a permanent magnet, a magnetic strip, or a magnetic slurry, or a combination thereof.

15. The system of claim 14, wherein the magnetic slurry includes nail polish.
